(19) United States
(12) Patent Application Publication
(10) Pub. No.: US 2023/0087975 A1
(43) Pub. Date: Mar. 23, 2023

(54) WATER-SOLUBLE CONJUGATED POLYMER FOR PHOTOTHERMAL THERAPY, POLYMERIZED MONOMER THEREOF, PREPARATION METHOD THEREFOR, AND APPLICATION THEREOF

(71) Applicant: SOUTH CHINA UNIVERSITY OF TECHNOLOGY, Guangzhou, Guangdong (CN)

(72) Inventors: Lei YING, Guangzhou (CN); Ting GUO, Guangzhou (CN)

(73) Assignee: SOUTH CHINA UNIVERSITY OF TECHNOLOGY, Guangzhou, Guangdong (CN)

(21) Appl. No.: 17/787,952

(22) PCT Filed: Sep. 30, 2020
(86) PCT No.: PCT/CN2020/119345

§ 371 Date: Jun. 22, 2022

(30) Foreign Application Priority Data
Dec. 31, 2019 (CN) 201911411861.8

(51) Int. Cl. A61K 41/00; A61K 47/59

(52) U.S. Cl. CPC A61K 41/0052; A61K 47/59

(57) ABSTRACT

A water-soluble conjugated polymer for photothermal therapy, a polymerized monomer thereof, a preparation method therefor, and application thereof. The water-soluble conjugated polymer has good solubility in an aqueous solution, has excellent biocompatibility, does not need to be subjected to coating treatment, can be directly used for photothermal therapy, is easy to use, has a nanometer size, and can enter cells easily. Polar groups are contained in side chains, and the water-soluble conjugated polymer is capable of targeting, can locate intracellular organelles, has excellent photostability and chemical properties as well as high photothermal conversion efficiency, can achieve photothermal therapy of near-infrared region I or II, with high treatment efficiency and few side effects. In the preparation method for the water-soluble conjugated polymer, raw materials can be easily obtained, synthesis conditions are mild, and the purification is convenient. The preparation method is simple, can be easily implemented, and has a great application prospect.

### CROSS-REFERENCE TO RELATED APPLICATIONS

This is the U.S. National Stage of International Patent Application No. PCT/CN2020/119345 filed on Sep. 30, 2020, which in turn claims the benefit of Chinese Patent Application No. 201911411861.8 filed on Dec. 31, 2019.

### FIELD OF THE INVENTION

The present invention belongs to the technical field of antitumor drugs, and specifically relates to a water-soluble conjugated polymer for photothermal therapy and its polymeric monomer, preparation method and application.

### BACKGROUND OF THE INVENTION

Cancer is one of the important diseases that threaten human health and lives. According to the World Cancer Report published by the World Health Organization in 2018, there were 18.1 million new cancer cases (9.5 million for males and 8.6 million for females) worldwide in 2018, with 9.6 million deaths (5.4 million for males and 4.2 million for females), further aggravating the global burden of cancer. Wherein, China, as a populous nation, accounts for a large part of the global cancer incidence and death. Therefore, cancer has become one of the major diseases that China must overcome independently.

Photothermal therapy can selectively heat cancer parts by means of photothermal materials under the action of light irradiation, so as to cause excessive heat generation inside cancer cells, thereby achieving the purpose of killing the cancer cells and inhibiting the tumor growth. Because cancer cells proliferate at a speed of much faster than that of normal tissues, the blood vessels inside the tumor tissues develop incompletely and the vascular walls are defective, which are less resistant to heat than the normal cellular tissues. When the temperature inside a cell reaches 40° C., the protein in the cells begins to deform; and when the temperature inside the cell reaches 50° C., irreversible damage will be caused. Using this point, photothermal treatment can damage cancer cells and destroy tumor tissues in the condition of without affecting normal cell tissues. Photothermal therapy has advantages of minimal invasiveness, less effect on normal cells and tissues, and fewer side effects. In recent years, photothermal therapy for cancer has gradually become a research hotspot for scientific researchers.

Through the continuous efforts of scientific researchers, the current photothermal materials are mainly divided into precious metal materials, carbon-based materials, transition metal compound nanomaterials, and organic photothermal materials. (1) Although precious metal materials (such as Au, Ag and the like) have high photothermal conversion efficiency, they have the shortages of poor metabolism in vivo, high cost, and certain toxic and side effects; (2) although carbon-based materials are non-toxic and have high photothermal conversion efficiency, they have weak absorption in the near-infrared band, which limits their further application; (3) transition metal compound two-dimensional materials have high photothermal conversion efficiency in the near-infrared region, but complicated preparation, larger size, being not easily absorbed by cells and slower metabolism etc. have become the bottleneck problem of their development; and (4) organic photothermal materials have the advantages of strong absorption in the near-infrared region, good biocompatibility, easy functionalization for the structure, short metabolic time in vivo, and so on. Therefore, relative to other types of photothermal materials, organic photothermal materials provide a new material system for photothermal therapy of tumor.

A biological system is a complex water-soluble environment. Organic photothermal materials containing oil-soluble groups have poor water solubility and high biological toxicity. However, water-soluble organic photothermal materials contain hydrophilic groups, which make the photothermal materials have better solubility in water and good biocompatibility, so that they have more advantages than oil-soluble organic photothermal materials in biological application. Therefore, it is of important practical significance to study and develop an organic photothermal material with a simple preparation method, high photothermal conversion efficiency and good biocompatibility.

### CONTENTS OF THE INVENTION

In order to overcome the above-described shortcomings and deficiencies in the prior art, the primary object of the present invention is to provide a polymeric monomer of water-soluble conjugated polymers for photothermal therapy.

Another object of the present invention is to provide a water-soluble conjugated polymer for photothermal therapy prepared by using the above-mentioned polymeric monomer. The conjugated polymer has excellent water solubility, photostability and photothermal conversion performance, and has huge application potential in the field of photothermal therapy for cancer.

Still another object of the present invention is to provide a method for preparing the above-mentioned water-soluble conjugated polymer for photothermal therapy.

Yet still another object of the present invention is to provide the application of the above-mentioned water-soluble conjugated polymer for photothermal therapy.

The objects of the present invention are achieved through the following technical solutions.

A polymeric monomer of water-soluble conjugated polymers for photothermal therapy, named as M, is provided, having the chemical structure as shown as formula (I):

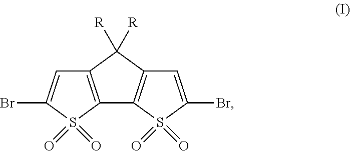

in the formula, R is —(CH₂)ₘ—X, where m is any integer from 1 to 10, and X is any one of the following structures: SO₃⁻, Ph₃P⁺, NH₄⁺, N(CH₂CH₃)₂ and the like.

The method for preparing the above-described polymeric monomer of water-soluble conjugated polymers for photothermal therapy comprises the following steps:

(1) subjecting 2,6-dibromo-4H-cyclopenta[2,1-b:3,4-b′]dithiophene to an alkylation reaction with 1,6-dibromohexane, under the actions of tetrabutylammonium iodide (TBAI) and a sodium hydroxide aqueous solution (NaOH (aq)), to obtain 2,6-dibromo-4,4-bis(6-bromohexyl)-4H-cyclopenta[2,1-b:3,4-b′]dithiophene;

(2) dissolving 2,6-dibromo-4,4-bis(6-bromohexyl)-4H-cyclopenta[2,1-b:3,4-b]dithiophene in anhydrous dichloromethane (DCM), and obtaining a compound 2,6-dibromo-4,4-bis(6-bromohexyl)-4H-cyclopenta[2,1-b:3,4-b′]dithiophene-1,1,7,7-tetraoxide, under the action of the oxidant m-chloroperoxybenzoic acid (m-CPBA); and

(3) dissolving 2,6-dibromo-4,4-bis(6-bromohexyl)-4H-cyclopenta[2,1-b:3,4-b′]dithiophene-1,1,7,7-tetroxide in toluene, and reacting with a monomer containing X to obtain the polymeric monomer M.

Further, in step (1), the molar ratio of 2,6-dibromo-4H-cyclopenta[2,1-b:3,4-b′]dithiophene, 1,6-dibromohexane, sodium hydroxide and tetrabutylammonium iodide is 1:2 to 5:3 to 8:0.05 to 0.1, preferably 1:2.5:5:0.1.

Further, the sodium hydroxide aqueous solution in step (1) is preferably a sodium hydroxide aqueous solution with a mass fraction of 40 to 60 wt %, and more preferably a sodium hydroxide aqueous solution with a mass fraction of 50 wt %.

Further, the alkylation reaction described in step (1) is preferably carried out at a condition of 80° C. to 120° C. for 20 to 30 hours; more preferably at 100° C. for 24 hours.

Further, in step (2), the molar ratio of 2,6-dibromo-4,4-bis(6-bromohexyl)-4H-cyclopenta[2,1-b:3,4-b′]dithiophene to m-chloroperoxybenzoic acid is 1:4 to 8, preferably 1:6.

Further, the reaction described in step (2) is preferably carried out at a condition of 20° C. to 30° C. for 10 to 15 hours, more preferably at 25° C. for 12 hours.

Further, in step (3), the molar ratio of 2,6-dibromo-4,4-bis(6-bromohexyl)-4H-cyclopenta[2,1-b:3,4-b′]dithiophene-1,1,7,7-tetroxide to the X-containing monomer is 1:2 to 5, preferably 1:2.5.

Further, the reaction described in step (3) is preferably carried out at a condition of 100° C. to 120° C. for 10 to 15 hours, more preferably at 110° C. for 12 hours.

The specific preparation route of the polymeric monomer M is as follows:

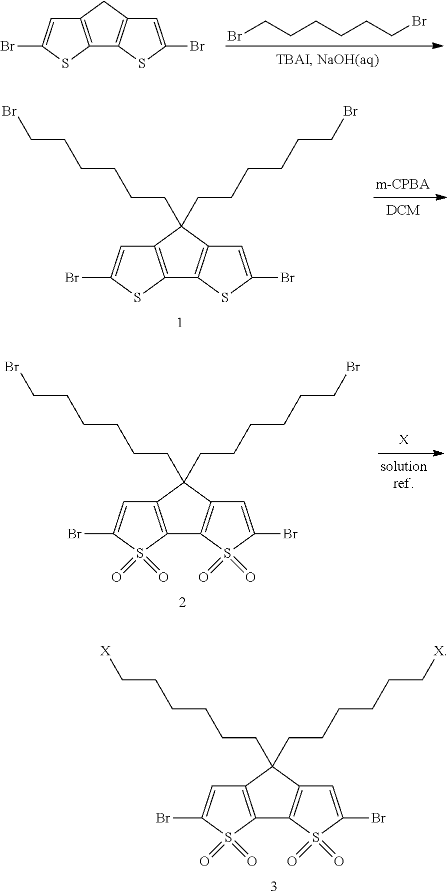

The chemical structure of the water-soluble conjugated polymer for photothermal therapy is shown as the following formula (II):

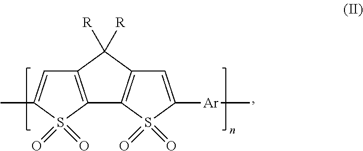

in this formula, the degree of polymerization n is any integer from 1 to 300;

having the same definition as in formula (I), R is —(CH₂)ₘ—X, where m is from 1 to 10, and X is any one of the following structures: SO₃⁻, Ph₃P⁺, NH₄⁺, N(CH₂CH₃)₂ and the like; and

the structural unit Ar is one of the following conjugated structural units:

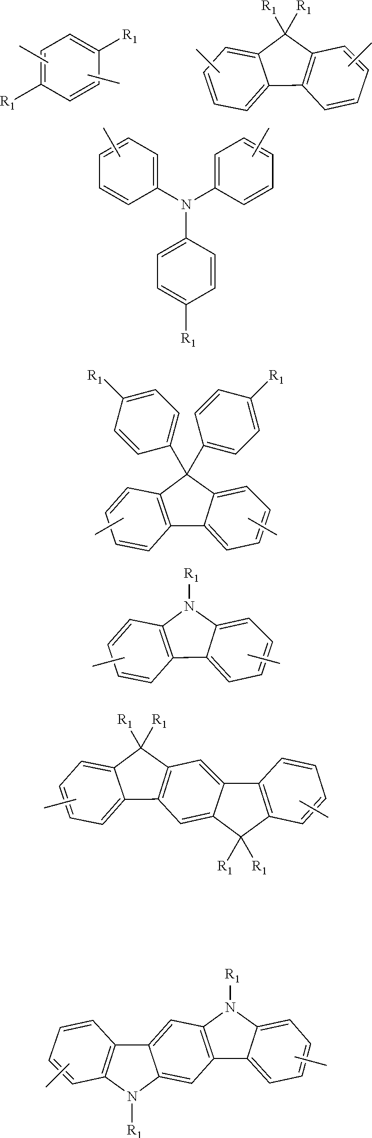

wherein, R₁ is H or a linear or branched alkyl group with 1-20 carbon atoms.

The preparation method of the above-described water-soluble conjugated polymer comprises the following steps: under the protection of inert gas, using an organic solvent to completely dissolve the polymeric monomer M and the monomer containing the Ar structural unit, heating to 60° C. to 100° C. to incur the Suzuki polymerization reaction under the actions of a catalyst and tetraethylammonium hydroxide, with the reaction time being 12 to 36 hours; adding phenylboronic acid, and continuing the reaction for 6 to 12 hours at a constant temperature; then adding bromobenzene, and continuing the reaction for 6 to 12 hours at a constant temperature; after the reaction stops, purifying the obtained reaction solution to obtain the target product.

Further, the organic solvent is preferably at least one of dimethyl sulfoxide, tetrahydrofuran and N,N-dimethylformamide.

Further, the catalyst is a system of palladium acetate and tricyclohexylphosphine or a system of tetrakis(triphenylphosphine)palladium, more preferably a system of palladium acetate and tricyclohexyl phosphine, and most preferably a catalytic system of palladium acetate and tricyclohexyl phosphine formulated at a molar ratio of 1:2.

Further, the purification is operated specifically as follows: the obtained reaction solution is cooled to room temperature and then poured into acetone for precipitation, and then the obtained precipitate is filtered and dried to obtain a crude product; the crude product is extracted successively with methanol, acetone and n-hexane, then dissolved by using deionized water, and settled out in an acetone solution for several times, filtered, and dried.

Further, the molar amounts of the polymeric monomer M and the monomer containing the Ar structural unit are equal; the amount of the catalyst is 5% to 5% by mol of the total amount of the reaction monomer; the amount of phenylboronic acid is 10% to 20% by mol of the total amount of the reaction monomer; and the amount of bromobenzene is 2 to 5 times the molar amount of phenylboronic acid.

The above-described water-soluble conjugated polymers for photothermal therapy are applied in the field of photothermal therapy.

The above-described water-soluble conjugated polymers for photothermal therapy are used in the preparation of a photothermal agent for photothermal therapy.

The water-soluble conjugated polymer material for photothermal therapy of the present invention has the following characteristics: Because of the hydrophilic group contained in its side chain, it has excellent solubility in an aqueous solution; it employs a unique molecular configuration, which is beneficial to broadening the absorption range of the conjugated polymer and achieving absorption in the near-infrared-II region; the main chain of the conjugated polymer contains an electron-deficient group, sulfuryl, which is beneficial to increasing the fluorescence quantum yield of the material and realizing the near-infrared imaging of the material; the side chain contains a hydrophilic group, has cell targeting properties, and can realize the localization of cancer cells, more conducive to the entry of the water-soluble conjugated polymers into tumors for photothermal therapy.

The present invention has the following advantages and beneficial effects compared to the prior art:

1. The photothermal material of the present invention is a kind of water-soluble polymer having big solubility in an aqueous solution, has excellent biocompatibility without the need for coating treatment, and can be directly used for photothermal therapy with easy operation; it has a nanometer size and is easy to enter cells.

2. The photothermal material of the present invention has the characteristics of being non-toxic to cells under the action of laser light, and having a high damaging effect on cancer cells under near-infrared light irradiation; its side chain contains polar groups, which are targeting and can locate the organelles in cells. Wherein, the cells are cancer cells, especially 4T1 cells.

3. Through reasonable structural design, the present invention enhances the intermolecular interaction, improves the non-radiative transition probability of the water-soluble conjugated polymer, and further improves its photothermal conversion efficiency, having better effect in the near-infrared photothermal therapy. The photothermal material of the present invention has excellent photostability and chemical properties, as well as high photothermal conversion efficiency; it has a wide absorption range, can realize the absorption response to the near-infrared-I region and the near-infrared-II region, and can go deeper into the lesion area to realize photothermal therapy, having high treatment efficiency and few side effects, thus having practical application prospects.

4. The preparation method of the photothermal material of the present invention has the advantages of easy availability of raw materials, mild synthesis conditions, simple preparation methods, and convenient purification.

### BRIEF DESCRIPTION OF THE DRAWINGS

FIG. 1 is a diagram of the Ultraviolet-visible absorption spectra of a water-soluble polymer HP1 and a contrast polymer HP11 in an aqueous solution.

FIG. 2 is a diagram of results for the cytotoxicity test of the water-soluble polymer HP1.

FIG. 3 is a diagram of the photothermal performance test of the water-soluble polymer HP1.

### DETAILED DESCRIPTION OF THE EMBODIMENTS

The present invention will be further described below in detail with reference to examples and drawings, but the embodiments of the present invention are not limited thereto.

### 1. Preparation of Polymeric Monomer (HM1)

### (1) Preparation of 2,6-dibromo-4,4-bis(6-bromohexyl)-4H-cyclopenta[2,1-b:3,4-b′]dithiophene (1)

2,6-dibromo-4H-cyclopenta[2,1-b:3,4-b′]dithiophene (6.68 g, 20 mmol), 1,6-dibromohexane (12.1 g, 50 mmol), a sodium hydroxide aqueous solution with a mass fraction of 50% (4 mL of 1 g/mL deionized water, 100 mmol) and tetrabutylammonium iodide (0.74 g, 2.0 mmol) were added into a 100 mL two-necked flask in an argon atmosphere, and subjected to reacting at 100° C. for 24 hours. After the reaction stopped, the reaction was quenched with water, and the following operations were performed: extracting with dichloromethane, drying with anhydrous magnesium sulfate, concentrating the solution, and then purifying by silica gel column chromatography with petroleum ether as the eluent, with a yield of 89%. Results for ¹H NMR, ¹³C NMR, MS and elemental analysis showed that the obtained compound was the target product. The chemical reaction equation of the preparation process is shown as follows:

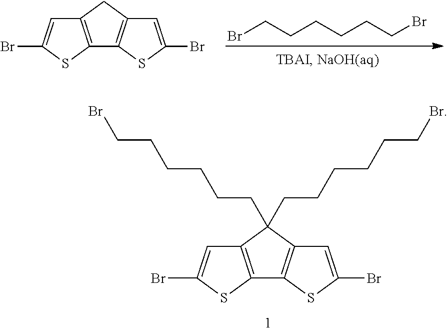

### (2) Preparation of 2,6-dibromo-4,4-bis(6-bromohexyl)-4H-cyclopenta[2,1-b:3,4-b′]dithiophene-1,1,7,7-tetroxide (2)

2,6-dibromo-4,4-bis(6-bromohexyl)-4H-cyclopenta[2,1-b:3,4-b]dithiophene (10.8 g, 20 mmol) and m-chloroperoxybenzoic acid (20.7 g, 120 mmol) were dissolved in 80 mL of anhydrous dichloromethane (DCM) in an argon atmosphere, and were subjected to reacting at 25° C. for 12 hours. After the reaction stopped, the reaction was quenched with water, and the following operations were performed: extracting with dichloromethane, drying with anhydrous magnesium sulfate, concentrating the solution, and then purifying by silica gel column chromatography with petroleum ether as the eluent, thus obtaining a white solid at a yield of 82%. Results for ¹H NMR, ¹³C NMR, MS and elemental analysis showed that the obtained compound was the target product. The chemical reaction equation of the preparation process is shown as follows:

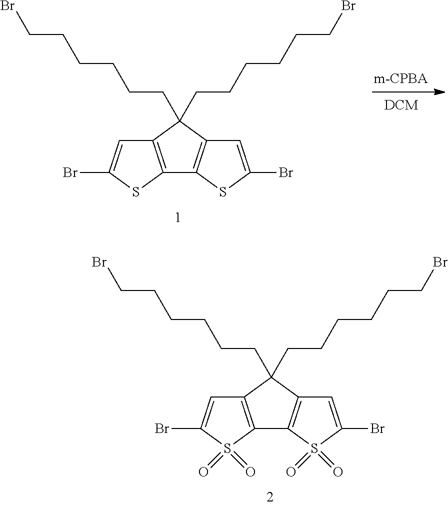 [SMILES: BrCCCCCCC1(CCCCCCBr)c2cc(Br)sc2-c2sc(Br)cc21 ; O=S1(=O)C(Br)=CC2=C1C1=C(C=C(Br)S1(=O)=O)C2(CCCCCCBr)CCCCCCBr]

### (3) Preparation of Monomer HM1

2,6-dibromo-4,4-bis(6-bromohexyl)-4H-cyclopenta[2,1-b:3,4-b′]dithiophene-1,1,7,7-tetroxide (10.8 g, 15 mmol), triphenylphosphine (PPh₃) (0.66 g, 37.5 mmol) and 50 mL of toluene were added into a 250 mL two-necked flask in an argon atmosphere, and were subjected to reacting at a temperature of 110° C. for 12 hours. After the reaction stopped, the reaction was quenched with water, and the following operations were performed: spin-drying to remove toluene, extracting with dichloromethane, drying with anhydrous magnesium sulfate, concentrating the solution, and then purifying by silica gel column chromatography with a mixed solvent of petroleum ether and dichloromethane (at a volume ratio of 1:1) as the eluent, thus obtaining a white solid at a yield of 92%. Results for ¹H NMR, ¹³C NMR, MS and elemental analysis showed that the obtained compound was the target product HM1. The chemical reaction equation of the preparation process is shown as follows:

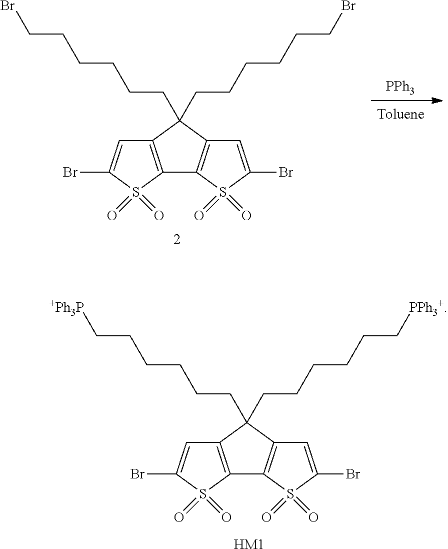

### (4) Preparation of Monomer HM2

2,6-dibromo-4,4-bis(6-bromohexyl)-4H-cyclopenta[2,1-b:3,4-b′]dithiophene-1,1,7,7-tetroxide (10.8 g, 15 mmol), diethylamine (NH(CH₂CH₃)₂) (2.7 g, 37.5 mmol) and 50 mL of toluene were added into a 250 mL two-necked flask in an argon atmosphere, and were subjected to reacting at 120° C. for 24 hours. After the reaction stopped, the reaction was quenched with water, and the following operations were performed: spin-drying to remove toluene, extracting with dichloromethane, drying with anhydrous magnesium sulfate, concentrating the solution, and then purifying by silica gel column chromatography with a mixed solvent of petroleum ether and dichloromethane (at a volume ratio of 1:1) as the eluent, thus obtaining a white solid at a yield of 75%. Results for ¹H NMR, ¹³C NMR, MS and elemental analysis showed that the obtained compound was the target product HM2. The chemical reaction equation of the preparation process is shown as follows:

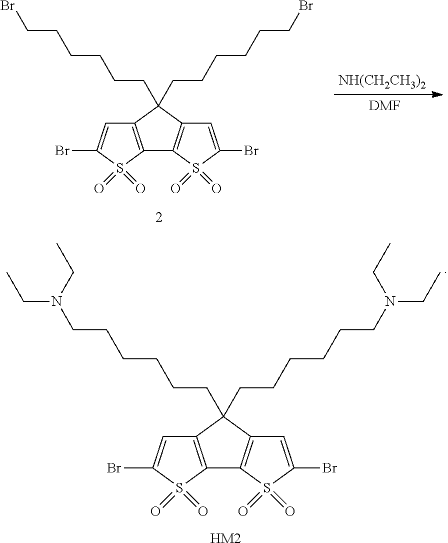

### (5) Preparation of Monomer HM3

2,6-dibromo-4,4-bis(6-bromohexyl)-4H-cyclopenta[2,1-b:3,4-b′]dithiophene-1,1,7,7-tetroxide (10.8 g, 15 mmol), triethyl phosphite (6.2 g, 37.5 mmol) and 50 mL of dichlorobenzene were added into a 250 mL two-necked flask in an argon atmosphere, and were subjected to reacting at a temperature of 130° C. for 24 hours. After the reaction stopped, the reaction was quenched with water, and the following operations were performed: extracting with dichloromethane, drying with anhydrous magnesium sulfate, concentrating the solution, and then purifying by silica gel column chromatography with a mixed solvent of petroleum ether and dichloromethane (at a volume ratio of 1:1) as the eluent, thus obtaining a white solid at a yield of 82%. Results for ¹H NMR, ¹³C NMR, MS and elemental analysis showed that the obtained compound was the target product HM3. The chemical reaction equation of the preparation process is shown as follows:

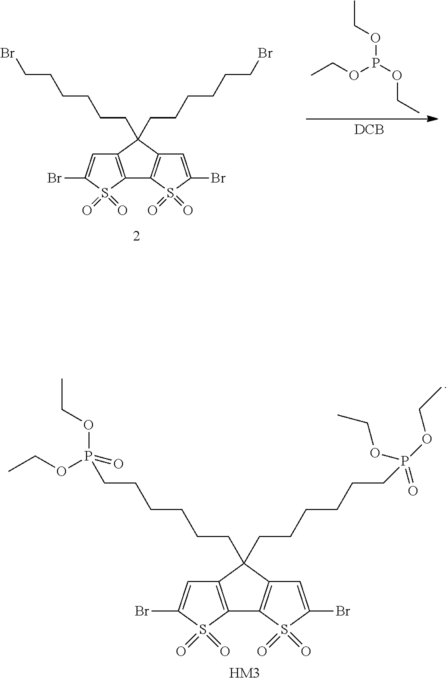

### (6) Preparation of Monomer HM4

2,6-dibromo-4,4-bis(6-bromohexyl)-4H-cyclopenta[2,1-b:3,4-b′]dithiophene-1,1,7,7-tetroxide (10.8 g, 15 mmol), trimethylamine (2.74 g, 37.5 mmol) and 50 mL of tetrahydrofuran were added into a 250 mL two-necked flask in an argon atmosphere, and were subjected to reacting at a temperature of 75° C. for 24 hours. After the reaction stopped, the reaction was quenched with water, and the following operations were performed: spin-drying to remove tetrahydrofuran, extracting with dichloromethane, drying with anhydrous magnesium sulfate, concentrating the solution, and then purifying by silica gel column chromatography, with a mixed solvent of petroleum ether and dichloromethane (at a volume ratio of 1:1) as the eluent, thus obtaining a white solid at a yield of 82%. Results for ¹H NMR, ¹³C NMR, MS and elemental analysis showed that the obtained compound was the target product HM4. The chemical reaction equation of the preparation process is shown as follows:

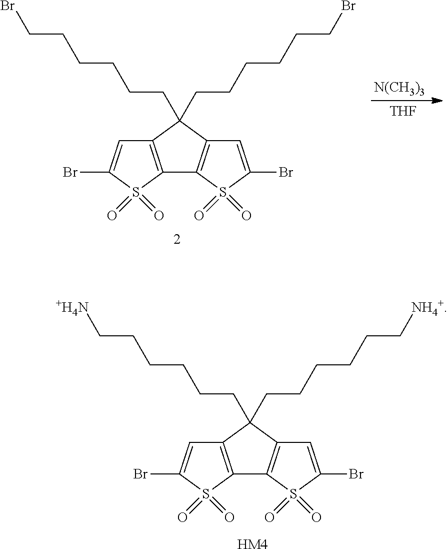

### (8) Preparation of Monomer HM5

2,6-dibromo-4,4-bis(6-bromohexyl)-4H-cyclopenta[2,1-b:3,4-b′]dithiophene-1,1,7,7-tetroxide (10.8 g, 15 mmol) and trifluoromethanesulfonic acid (CF₃HSO₃) (5.6 g, 37.5 mmol) were added into a 250 mL two-necked flask in an argon atmosphere, and were subjected to reacting at a temperature of 25° C. for 24 hours. After the reaction stopped, the reaction was quenched with water, and the following operations were performed: extracting with dichloromethane, drying with anhydrous magnesium sulfate, concentrating the solution, and then purifying by silica gel column chromatography with a mixed solvent of petroleum ether and dichloromethane (at a volume ratio of 1:1) as the eluent, thus obtaining a white solid at a yield of 85%. Results for ¹H NMR, ¹³C NMR, MS and elemental analysis showed that the obtained compound was the target product HM5. The chemical reaction equation of the preparation process is shown as follows:

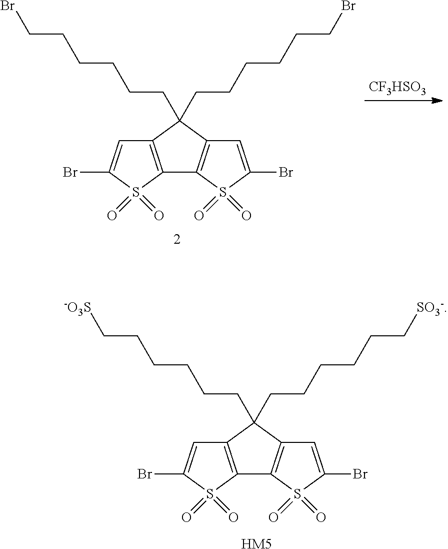

### (9) Preparation of Water-Soluble Polymer HP1

In an argon atmosphere, 1,4-benzenediboronic acid (83.0 mg, 0.50 mmol) and the monomer HM1 (413.0 mg, 0.50 mmol) were added into a 50 mL two-necked flask, with 12 mL of refined toluene, palladium acetate (2.80 mg, 12.45 μmol) and tricyclohexyl phosphine (6.98 mg, 24.90 μmol), and 3 mL of tetraethylammonium hydroxide added thereafter, and then were subjected to reacting for 24 hours after the temperature was raised to 80° C. Then, 20 mg of phenylboronic acid was added for end-capping. 12 hours later, 0.3 mL of bromobenzene was used for end-capping. After continuing the reaction for 12 hours, the reaction was stopped. While the temperature decreased to room temperature, the product was added dropwise into 300 mL of acetone for settling out and filtration. Then, the crude product was dissolved in 20 mL of acetone, and subjected to the following operations: column chromatography with silica gel having 200 to 300 meshes as the stationary phase and acetone as the eluent, settling out again in acetone after concentrating the solvent, stirring, filtering, and vacuum-drying to obtain a polymer solid. Finally, extracting was performed successively with methanol, acetone and n-hexane respectively for 24 hours to remove small molecules. Then, the solid was dissolved in deionized water, with acetone added dropwise for settling out, and the water-soluble polymer HP1 was obtained after vacuum-drying. Results for ¹H NMR, GPC and elemental analysis showed that the obtained compound was the target product. The chemical reaction equation of the preparation process is shown as follows:

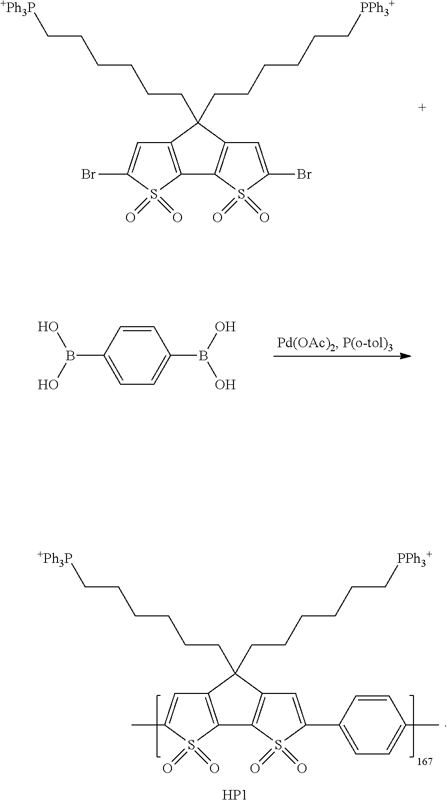

The cytotoxicity of the water-soluble polymer HP1 was detected by the CCK-8 method. The specific steps were as follows:

(1) Dissolving the water-soluble polymer HP1 in a phosphate buffer solution (PBS, containing 0.68 g of potassium dihydrogen phosphate and 316 mg of sodium hydroxide per 100 mL of the PBS solution) with pH=7.4 to a concentration of 1.0 mg/mL. and then diluting with a complete medium (DMEM containing 10% fetal bovine serum, 100 U/mL penicillin and 100 μg/mL streptomycin) to a concentration of 100 μg/mL, 200 μg/mL, 300 μg/mL, 400 μg/mL and 500 μg/mL, respectively;

(2) digesting 4T1 cells (breast cancer cells of mouse, ATCC) in the logarithmic growth phase with 0.25% trypsin, and uniformly diluting the cells to a concentration of 5×10⁴ cells/mL;

(3) adding the cell solution into 96-well plates at 100 μL per well, shaking the plate gently to be uniform, and then putting the plates into an incubator at 37° C. with 5% CO2 to incubate for 24 hours;

(4) adding the complete medium containing different concentrations of the water-soluble polymer HP1 into the 96-well plates at 100 μL per well, with 10 wells for each concentration, a total of 2 groups with one group having every 5 wells, i.e. an illuminated group and a non-illuminated group, with 0 μg/mL set as the control group, and putting the 96-well plates into the incubator to incubate for 12 hours;

(5) taking out the 96-well plate of the illuminated group, illuminating it with 808 nm laser (with a power of 0.5 W/cm²) for 5.0 minutes, and then putting it into the incubator and continuing to incubate for 12 hours; while the 96-well plate of the non-illuminated group being directly cultivated for 24 hours without a need to receive illumination treatment;

(6) washing out the medium waste liquid in the 96-well plates of the illuminated group and the non-illuminated group, adding 100 μL of a complete medium containing 10% CCK-8 into each well, and then putting the 96-well plates back into the incubator to incubate for 1 hours;

(7) putting the 96-well plates of the illuminated group and the non-illuminated group into a microplate reader, with the absorption peak tested at 450 nm, then measuring the absorbance of each well, calculating the average value and standard deviation for the absorbance of 5 wells of each group, and calculating the survival rate of the cells, with results for the CCK-8 test shown as FIG. 2.

It can be known from FIG. 2 that the survival rate of the 4T1 cells could be maintained 80% or more without illumination upon the water-soluble polymer HP1 at different concentrations, indicating that the water-soluble polymer HP1 had no cytotoxicity on condition of no illumination. Under illumination, however, the survival rate of the cells was related to the concentration of the water-soluble polymer; the greater the concentration of HP1 was, the lower the survival rate of the cells would be. At a concentration of 100 μg/mL, HP1 could kill 16% of the 4T1 cells; at a concentration of 200 μg/mL, HP1 could kill 28% of the 4T1 cells; at a concentration of 300 μg/mL, HP1 could kill 43% of the 4T1 cells; at a concentration of 400 μg/mL, HP1 could kill 60% of the 4T1 cells; and at a concentration of 500 μg/mL, HP1 could kill 77% of the 4T1 cells. These results indicated that the water-soluble polymer HP1 had an excellent photothermal therapeutic effect on the 4T1 cells.

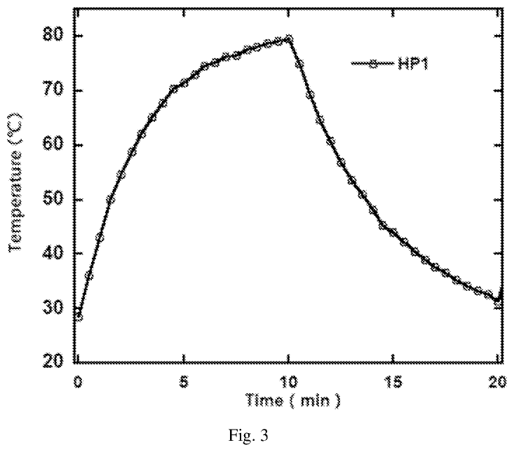

The photothermal performance of the water-soluble polymer HP1 was tested by an 808 nm laser. The water-soluble polymer HP1 with a concentration of 100 μg/mL was placed under a laser light source with a wavelength of 808 nm and a power of 1.0 W/cm², and then the temperature of the HP1 aqueous solution was recorded every 30 seconds. After the water-soluble polymer HP1 was illuminated for 10 minutes, the light source was removed, and the temperature of the HP1 aqueous solution was allowed to drop down naturally and recorded every 30 s. The temperature-rising and temperature-falling curves for the water-soluble polymer HP1 are shown as FIG. 3.

It can be known from FIG. 3 that the temperature of HP1 rose continuously under the illumination of the 808 nm laser light, rising faster at the beginning of the illumination and then tending to be rise gently. When the HP1 aqueous solution was not illuminated, its temperature was 28.5° C.; its temperature rose to 36.2° C. after 30 seconds of illumination; and the temperature eventually rose to 79.5° C. after 10 minutes of illumination. After removal of the light source, the temperature of the HP1 aqueous solution quickly decreased; and the temperature dropped to 75° C. in 30 seconds after removal of the light source. According to the cooling curve, the photothermal conversion efficiency of HP1 was calculated to be 53.2%. This indicates that this polymer has excellent photothermal conversion efficiency, can have excellent effects in photothermal therapy, and is thus a kind of photothermal material with application prospects.

The water-soluble polymers HP2 to HP10 were synthesized, respectively. The raw materials required by the reactions, reaction products, and yields are shown as Table 1 below. Reference was made to the synthesis method for the water-soluble polymer HP1, except that the monomer HM1 and 1,4-benzenediboronic acid were replaced with raw materials 1 and 2, respectively.

TABLE 1
Raw materials, reaction products and yields for synthesizing polymers HP2 to HP10

| Polymer | | | | |
|---|---|---|---|---|
| name | Raw material 1 | Raw material 2 | products | Yield |
| HP2 | 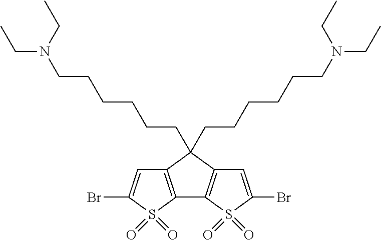 [SMILES: CCN(CC)CCCCCCC1(CCCCCCN(CC)CC)C2=C(C3=C1C=C(Br)S3(=O)=O)S(=O)(=O)C(Br)=C2] | 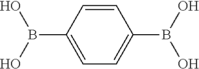 [SMILES: OB(O)c1ccc(B(O)O)cc1] | 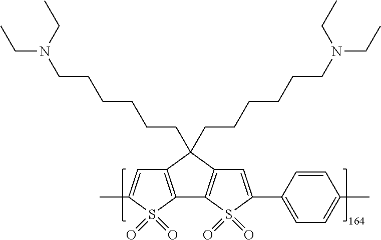 | 89% |
| HP3 | 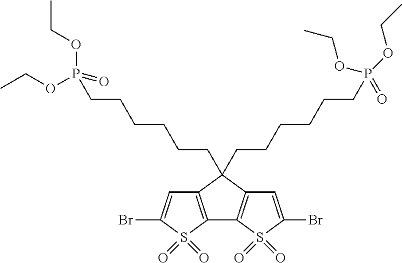 [SMILES: CCOP(=O)(CCCCCCC1(CCCCCCP(=O)(OCC)OCC)C2=C(C3=C1C=C(Br)S3(=O)=O)S(=O)(=O)C(Br)=C2)OCC] | 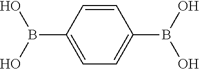 [SMILES: OB(O)c1ccc(B(O)O)cc1] | 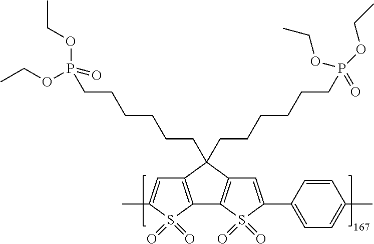 | 87% |
| HP4 | 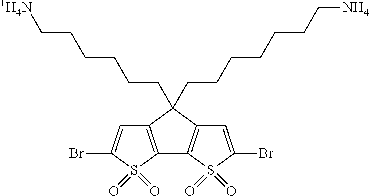 [SMILES: [NH3+]CCCCCCCC1(CCCCCC[NH3+])C2=C(C3=C1C=C(Br)S3(=O)=O)S(=O)(=O)C(Br)=C2] | 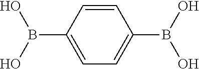 [SMILES: OB(O)c1ccc(B(O)O)cc1] | 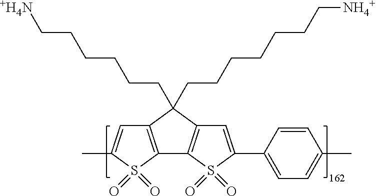 | 79% |
| HP5 | 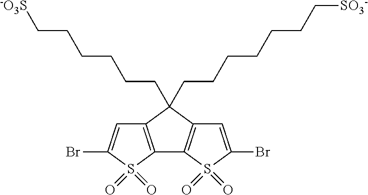 | 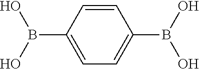 [SMILES: OB(O)c1ccc(B(O)O)cc1] | 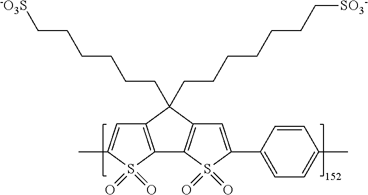 | 83% |
| HP6 | 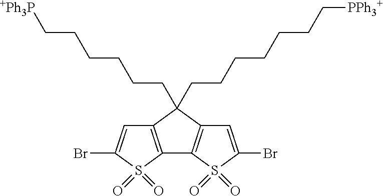 | 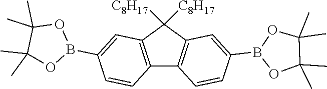 | 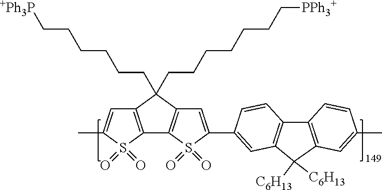 | 67% |
| HP7 | 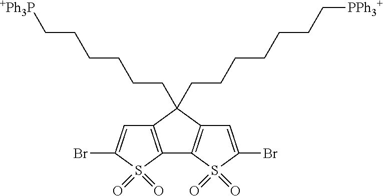 | 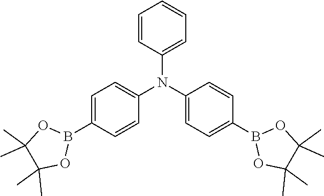 | 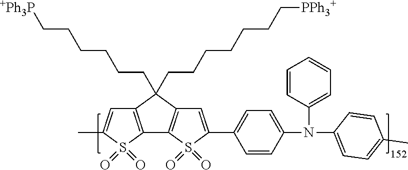 | 54% |
| HP8 | 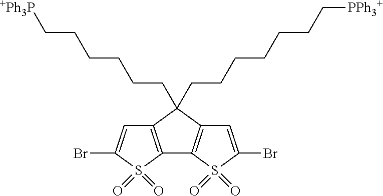 | 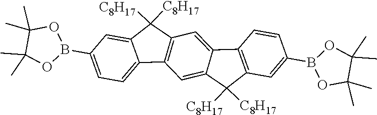 | 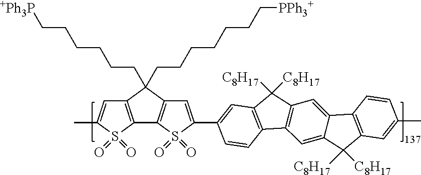 | 65% |
| HP9 | 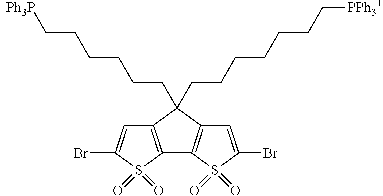 | 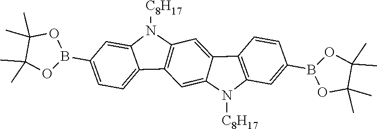 | 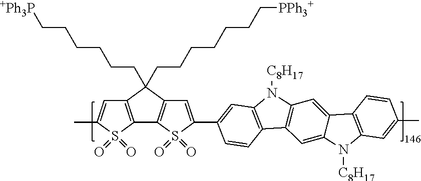 | 73% |
| HP10 | 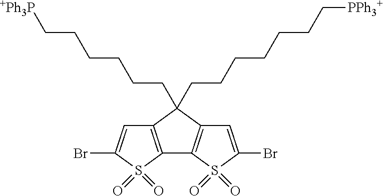 | 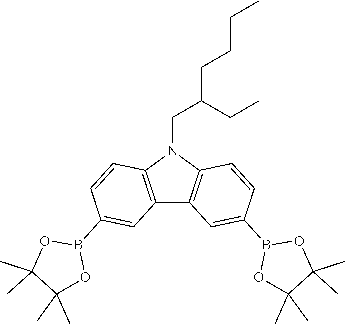 | 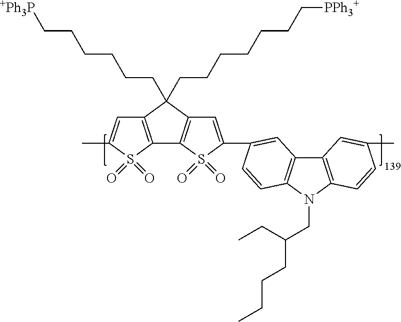 | 76% |

The cytotoxicity of the water-soluble polymers HP2 to HP10 was detected by the CCK-8 method with reference to the water-soluble polymer HP1. It was found through experiments that, when the solutions of HP2 to HP10 were in the concentration range of 0 to 500 μg/mL, the survival rate of the 4T1 cells was higher than 80% under the condition of no laser light illumination, which exhibited excellent biocompatibility. Under a condition for treatment of the illumination of the 808 nm laser light with a power of 1.0 W/cm², when the concentration of HP2 to HP10 was 500 μg/mL; HP2 could kill 82% of the 4T1 cells HP3 could kill 88% of the 4T1 cells; HP4 could kill 81% of the 4T1 cells; HP5 could kill 84% of the 4T1 cells; HP6 could kill 86% of the 4T1 cells; HP7 could kill 89% of the 4T1 cells; HP8 could kill 82% of the 4T1 cells; HP9 could kill 76% of the 4T1 cells; and HP10 could kill 83% of the 4T1 cells.

Through the tests of the temperature-rising and temperature-falling curves, the photothermal conversion efficiency of the water-soluble polymers HP2 to HP10 was 59.3%, 62.0%, 68.1%, 69.5%, 70.9%, 71.8%, 78.3%, 77.4% and 79.8%, respectively. The higher photothermal conversion efficiency indicates that the water-soluble polymers claimed by this patent application have excellent application prospects for photothermal therapy.

### (10) Preparation of Contrast Polymer HP11

### (1) Preparation of Compound DM1

p-Diphenol (11 g, 0.10 mol), tetrabutylammonium bromide (0.32 g, 1.00 mmol), 50 wt % sodium hydroxide aqueous solution (20 mL of 1 g/mL deionized water) and toluene solvent (200 mL) were added into a 500 mL two-necked flask, under the protection of argon. Upon heating and stirring. 1,6-dibromohexane (53.45 g, 0.22 mmol) was added when the temperature was stabilized at 80° C. After reacting for 6 hours, the reaction was terminated, the organic layer was separated and concentrated, and then the crude product was purified by a method of column chromatography with petroleum ether as the eluent, thus finally obtaining 25.1 g of a white solid at a yield of 91%. Results for ¹H NMR, ¹³C NMR, MS and elemental analysis showed that the obtained compound was the target product DM1. The chemical reaction equation is shown as follows:

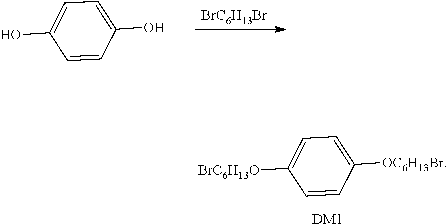

### (2) Preparation of Compound DM2

Under a dark condition, the compound DM1 (15.65 g, 35.9 mmol) and carbon tetrachloride (150 mL) were added into a 250 mL single-necked reaction flask, and then liquid bromine (12.6 g, 79.0 mmol) was successively added in three parts. After reacting for 8 hours, the following operations were performed: adding a saturated sodium bisulfite solution, extracting with dichloromethane, collecting the organic phase, concentrating, and then recrystallizing the crude product with ethanol, to obtain 13.47 g of a white acicular crystal with a yield of 86%. Results for ¹H NMR, ¹³C NMR, MS and elemental analysis showed that the obtained compound was the target product. The chemical reaction equation is shown as follows:

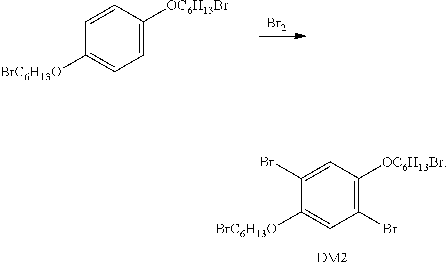

### (3) Preparation of Compound DM3

The compound DM2 (20.36 g, 34.4 mmol) and 100 mL of anhydrous tetrahydrofuran were added into a 250 mL three-necked flask. 2.4 M n-butyl lithium/n-hexane solution (35.8 mL, 86 mmol) was added dropwise at −78° C. under the protection of argon, and stirred for 2 hours at −78° C. Then, 2-isopropoxy-4,4,5,5-tetramethyl-1,3,2-ethylenedioxy borate (19.3 mL, 96.3 mmol) quickly added, and stirring was continued for 1.5 hours at −78° C. The reaction mixture gradually raised to room temperature, and were subjected to reacting for 10 hours while stirring. The following operations were performed: spin-drying the reaction solution, extracting with ethyl acetate, washing with a NaCl aqueous solution, drying with anhydrous magnesium sulfate, concentrating, and then purifying the crude product by silica gel column chromatography with petroleum ether/dichloromethane (3:1) as the eluent, thus obtaining 10.2 g of a white solid at a yield of 56%. Results for ¹H NMR, ¹³C NMR, MS and elemental analysis showed that the obtained compound was the target product. The chemical reaction equation is shown as follows:

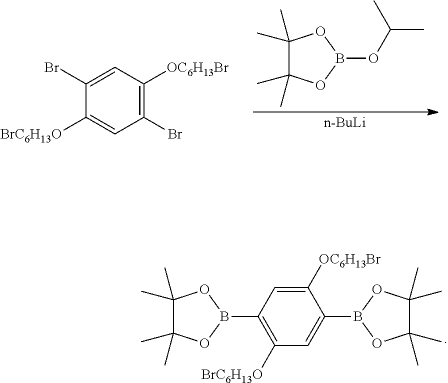

### (4) Preparation of Compound DM4

The compound DM3 (34.5 g, 50 mmol), 1-bromo-2-methylsulfinyl benzene (27.24 g, 125 mmol), tetrabutylammonium bromide (0.805 g, 2.5 mmol), tetrakis(triphenylphosphine) palladium (2.89 g, 2.5 mmol) and 100 mL of toluene solvent were sequentially added into a 250 mL three-necked flask under the protection of argon. stirred and heated to 110° C. 50 wt % potassium carbonate aqueous solution (24.6 mL, 125 mmol) was added to react for 24 hours. The solvent was spin-dried, and the crude product was purified by silica gel column chromatography with petroleum ether/dichloromethane (4:1) as the eluent, thus obtaining 10.2 g of a light yellow viscous liquid at a yield of 74%. Results for ¹H NMR, ¹³C NMR, MS and elemental analysis showed that the obtained compound was the target product. The chemical reaction equation is shown as follows:

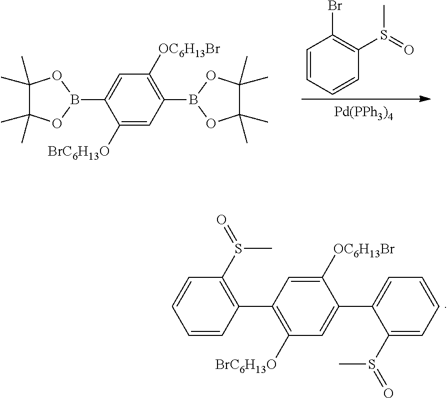

### (5) Preparation of Compound DM5

The compound DM4 (8.6 g, 15.5 mmol) was added into 10 mL of trifluoromethane sulfonic acid at a temperature of 25° C., and stirred for 20 hours at a condition of room temperature, and then the reaction solution was added dropwise into ice water. Then, the reaction solution was subjected to suction-filtering to obtain a light yellow solid powder, and the solid powder was dried in the air. The light yellow solid powder was added into 100 mL of pyridine, and heated for reflux for 6 hours with the introduction of nitrogen gas. The reaction was terminated with the temperature cooled to room temperature thereafter, and extracting and neutralizing the excess pyridine with hydrochloric acid were carried out. Column chromatography was performed with pure petroleum ether, and recrystallizing was performed with ethanol, to obtain a yellow powdery solid (2.43 g, 32%). Results for ¹H NMR, ¹³C NMR, MS and elemental analysis showed that the obtained compound was the target product. The chemical reaction equation is shown as follows:

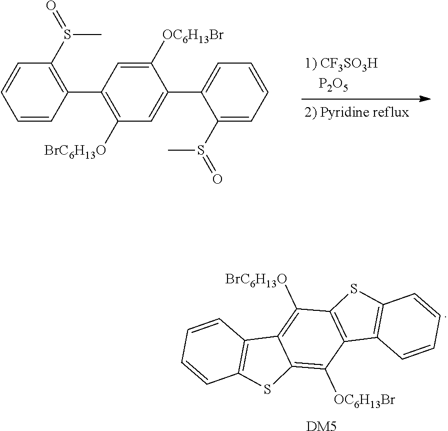

### (6) Preparation of Compound DM6

The compound DM5 (2.38 g, 3.67 mmol) and iodine (46 mg, 0.18 mmol) were added into 40 mL of dichloromethane, and liquid bromine (1.29 g, 8.07 mmol) was added dropwise under dark conditions to react for 10 hours. A saturated sodium bisulfite aqueous solution was added into the reaction system. When the system became colorless, the organic phase was separated and concentrated. The crude product was purified by a method of column chromatography with petroleum ether as the eluent, finally obtaining 1.55 g of a yellow solid at a yield of 65%. Results for ¹H NMR, ¹³C NMR, MS and elemental analysis showed that the obtained compound was the target product. The chemical reaction equation is shown as follows:

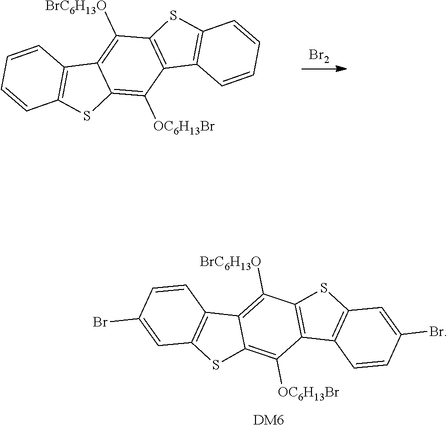

### (7) Preparation of Compound DM7

The compound DM6 (1.49 g, 1.85 mmol) and 3-chloroperoxybenzoic acid (3.4 g, 20 mmol) were dissolved in 120 mL of dichloromethane, and subjected to reacting for 5 hours under stirring. The reaction solution was poured into a cold sodium hydroxide aqueous solution with a concentration of 10% by mass and stirred for 30 min. The organic layer was washed with water three times, and the organic phase was collected, concentrated, and purified by chromatography column with dichloromethane/ethyl acetate (at a volume ratio of 1:2) as the eluent, and then recrystallized with ethanol to obtain a yellow solid (1.12 g, 85%). Results for ¹H NMR, ¹³C NMR, MS and elemental analysis showed that the obtained compound was the target product. The chemical reaction equation is shown as follows:

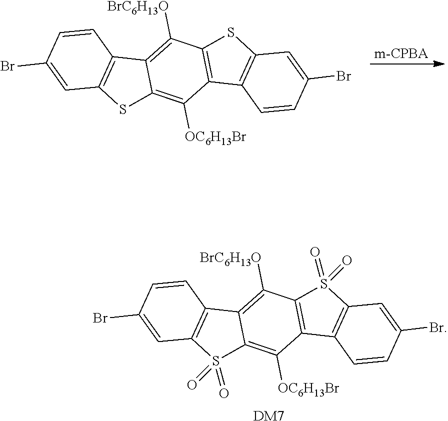

### (8) Preparation of Compound DM8

The compound DM7 (13.02 g, 15 mmol), triphenylphosphine (0.66 g, 37.5 mmol) and 50 mL of toluene were added into a 250 mL two-necked flask in an argon atmosphere, and were subjected to reacting for 12 hours at 110° C. After the reaction stopped, the reaction was quenched with water, and the following operations were performed: spin-drying to remove toluene, extracting with dichloromethane, drying with anhydrous magnesium sulfate, concentrating the solution, and then purifying by silica gel column chromatography with a mixed solvent of petroleum ether and dichloromethane (at a volume ratio of 1:1) as the eluent, thus obtaining a white solid at a yield of 92%. Results for ¹H NMR, ¹³C NMR, MS and elemental analysis showed that the obtained compound was the target product DM8. The chemical reaction equation of the preparation process is shown as follows:

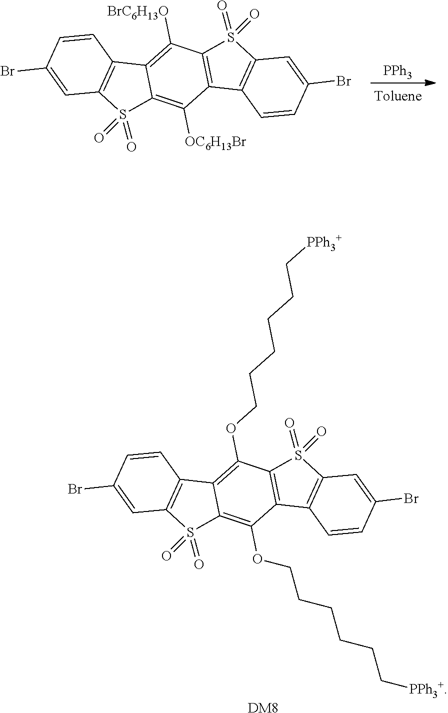

In an argon atmosphere, 1,4-benzenediboronic acid (83.0 mg, 0.50 mmol) and the monomer DM8 (485.0 mg, 0.50 mmol) were added into a 50 mL two-necked flask, and then 12 mL of refined toluene, palladium acetate (2.80 mg, 12.45 μmol) and tricyclohexyl phosphine (6.98 mg, 24.90 μmol), 3 mL of tetraethylammonium hydroxide were added in sequence. The temperature was raised to 80° C., and the reaction lasted for 24 hours. 20 mg of phenylboronic acid was added for end-capping, and 0.3 mL of bromobenzene was also used for end-capping 12 hours later. After continuing the reaction for 12 hours, the reaction was terminated. While the temperature decreased to room temperature, the product was added dropwise into 300 mL of acetone for settling out, and the crude product was dissolved in 20 mL of acetone after filtration, and then subjected to column chromatography with silica gel having 200 to 300 meshes as the stationary phase and acetone as the eluent. Thereafter, the following operations were performed: concentrating the solvent, settling out again in acetone, stirring, filtering, and vacuum-drying, to obtain a polymer solid. Finally, extracting was performed successively with methanol, acetone and n-hexane for 24 h to remove small molecules. Then, the solid was dissolved in deionized water, and added dropwise into acetone for settling out, and then vacuum-dried, to obtain the contrast polymer HP11. Results for ¹H NMR, GPC and elemental analysis showed that the obtained compound was the target product. The chemical reaction equation of the preparation process is shown as follows:

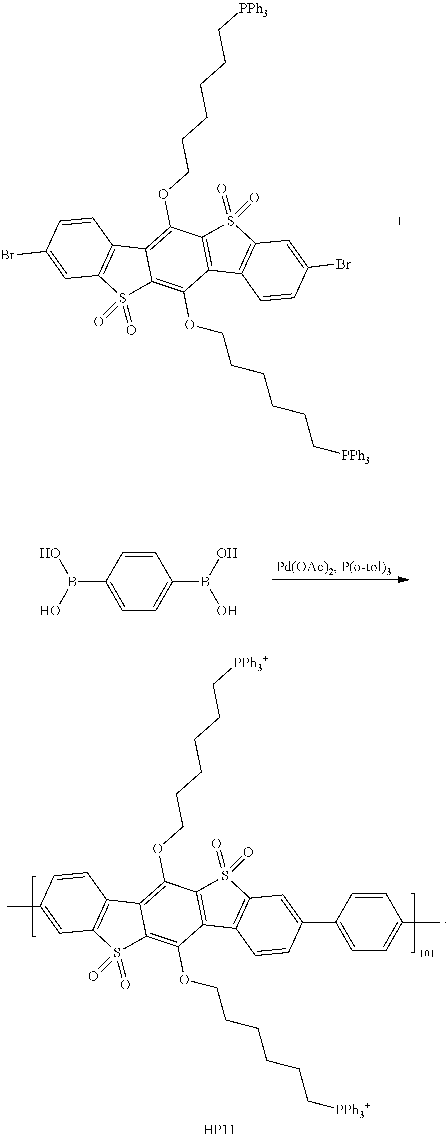

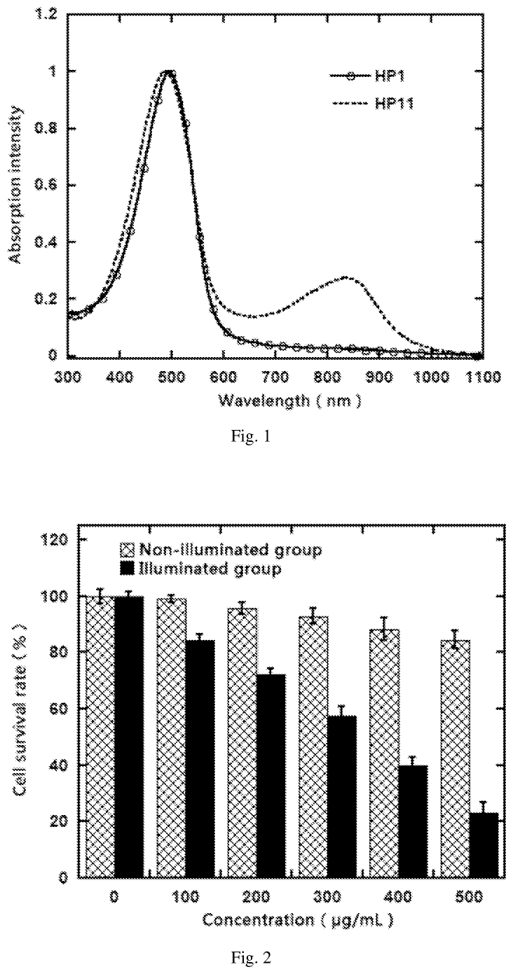

An Ultraviolet-visible spectrophotometer (UV-2400) from Shimadzu was used to collect the Ultraviolet-visible absorption spectra of the water-soluble polymer HP1 and the contrast polymer HP11 in the aqueous solution, with their concentrations of 50 μg/mL, and the results are shown as FIG. 1. It can be known from FIG. 1 that HP1 had the strongest UV absorption peak at 491 nm, the largest peak at 837 nm, and the absorption edge at 1075 nm, and can realize the absorption response to the near-infrared-II region. It can be inferred that HP1 had a deeper penetration depth. HP11 had the strongest UV absorption peak at 484 nm, and the absorption edge at 622 nm, with the light-absorption region in the visible light region. By contrast, HP1 and HP11 have the same Ar structure, but different monomers copolymerizing with Ar, which are

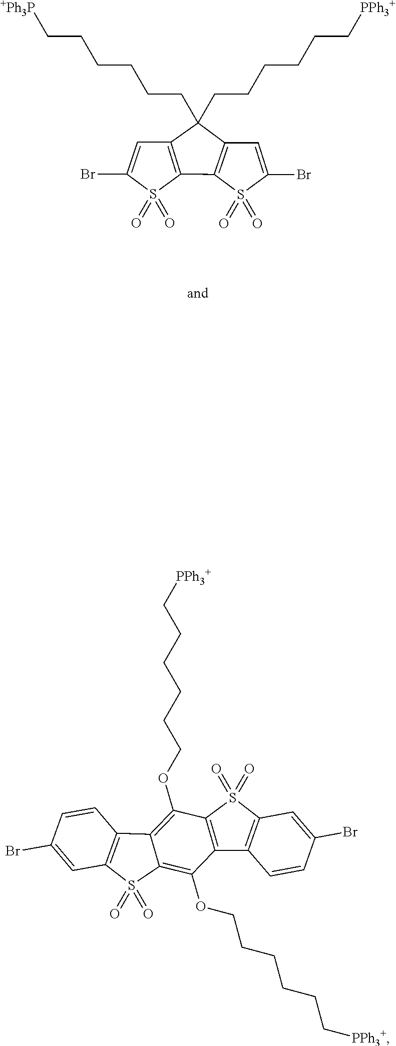

respectively. Comparing these two structures, they are both multi-membered fused ring structures containing two sulfone groups (—SO₂—), and their side chains have the same polarity. However, there was a difference that the absorption range of the polymer HP1 was much larger than that of HP11. At present, the light source that can be used for photothermal therapy is in the near-infrared region with a wavelength range of 700 to 1100 nm, which went beyond the absorption range of the polymer HP11, while the absorption range of the polymer HP1 was within this wavelength range. Therefore, the polymer HP11 cannot be used for photothermal therapy, while the polymer HP1 can be used for photothermal therapy.

The above examples are preferred embodiments of the present invention, but the embodiments of the present invention are not limited thereto, and any other alterations, modifications, replacements, combinations and simplifications made without departing from the spirit and principle of the present invention shall all be equivalent substitutions and included in the scope of protection of the present invention.

1. A polymeric monomer of water-soluble conjugated polymers for photothermal therapy, characterized in that: this polymeric monomer is named as M, and its chemical structure is shown as formula (I): 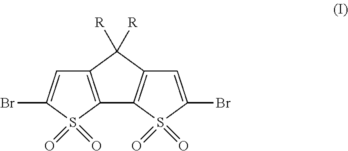
in the formula, R is —(CH₂)ₘ—X, where m is any integer from 1 to 10, and X is any one of the following structures: SO₃⁻, Ph₃P⁺, NH₄⁺ and N(CH₂CH₃)₂.

2. A method for preparing the polymeric monomer of water-soluble conjugated polymers for photothermal therapy according to claim 1, characterized in that, this method comprises the following steps:
(1) subjecting 2,6-dibromo-4H-cyclopenta[2,1-b:3,4-b′ ]dithiophene to an alkylation reaction with 1,6-dibromohexane, under the actions of tetrabutylammonium iodide and a sodium hydroxide aqueous solution, to obtain 2,6-dibromo-4,4-bis(6-bromohexyl)-4H-cyclopenta[2,1-b:3,4-b′]dithiophene;
(2) dissolving 2,6-dibromo-4,4-bis(6-bromohexyl)-4H-cyclopenta[2,1-b:3,4-b′ ]dithiophene in anhydrous dichloromethane, and obtaining a compound 2,6-dibromo-4,4-bis(6-bromohexyl)-4H-cyclopenta[2,1-b:3,4-b′]dithiophene-1,1,7,7-tetraoxide, under the action of an oxidant m-chloroperoxybenzoic acid; and
(3) dissolving 2,6-dibromo-4,4-bis(6-bromohexyl)-4H-cyclopenta[2,1-b:3,4-b′]dithiophene-1,1,7,7-tetroxide in toluene, and reacting with a monomer containing X to obtain the polymeric monomer M.

3. The method for preparing the polymeric monomer of water-soluble conjugated polymers for photothermal therapy according to claim 2, characterized in that:
in step (1), the molar ratio of 2,6-dibromo-4H-cyclopenta[2,1-b:3,4-b′]dithiophene, 1,6-dibromohexane, tetrabutylammonium iodide and sodium hydroxide is 1:2 to 5:3 to 8:0.05 to 0.1;
the sodium hydroxide aqueous solution in step (1) is a sodium hydroxide aqueous solution with a mass fraction of 40 to 60 wt %;
the alkylation reaction in step (1) is carried out at a condition of 80° C. to 120° C. for 20 to 30 hours;
in step (2), the molar ratio of 2,6-dibromo-4,4-bis(6-bromohexyl)-4H-cyclopenta[2,1-b:3,4-b′]dithiophene to m-chloroperoxybenzoic acid is 1:4 to 8;
the reaction described in step (2) is carried out at a condition of 20° C. to 30° C. for 10 to 15 hours;
in step (3), the molar ratio of 2,6-dibromo-4,4-bis(6-bromohexyl)-4H-cyclopenta[2,1-b:3,4-b′]dithiophene-1,1,7,7-tetroxide to the X-containing monomer is 1:2 to 5; and
the reaction described in step (3) is carried out at a condition of 100° C. to 120° C. for 10 to 15 hours.

4. The method for preparing the polymeric monomer of water-soluble conjugated polymers for photothermal therapy according to claim 2, characterized in that:
in step (1), the molar ratio of 2,6-dibromo-4H-cyclopenta[2,1-b:3,4-b′]dithiophene, 1,6-dibromohexane, tetrabutylammonium iodide and sodium hydroxide is 1:2.5:5:0.1;
the sodium hydroxide aqueous solution in step (1) is a sodium hydroxide aqueous solution with a mass fraction of 50 wt %;
the alkylation reaction in step (1) is carried out at a condition of 100° C. for 24 hours;
in step (2), the molar ratio of 2,6-dibromo-4,4-bis(6-bromohexyl)-4H-cyclopenta[2,1-b:3,4-b′]dithiophene to m-chloroperoxybenzoic acid is 1:6;
the reaction described in step (2) is carried out at a condition of 25° C. for 12 hours;
in step (3), the molar ratio of 2,6-dibromo-4,4-bis(6-bromohexyl)-4H-cyclopenta[2,1-b:3,4-b′]dithiophene-1,1,7,7-tetroxide to the X-containing monomer is 1:2.5; and
the reaction described in step (3) is carried out at a condition of 110° C. for 12 hours.

5. A water-soluble conjugated polymer for photothermal therapy, characterized in that, its chemical structure is shown as formula (II): 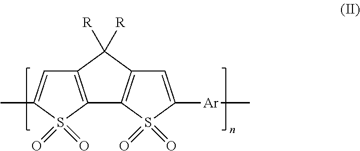
in this formula, the degree of polymerization n is any integer from 1 to 300;
R is —(CH₂)ₘ—X, where m is from 1 to 10, and X is any one of the following structures: SO₃⁻, Ph₃P⁺, NH₄⁺ and N(CH₂CH₃)₂;
the structural unit Ar is one of the following conjugated structural units: 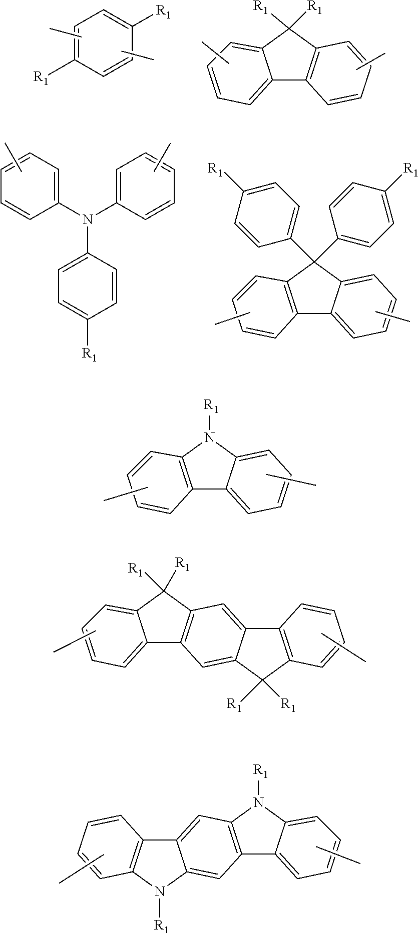
wherein, R₁ is H or a linear or branched alkyl group with 1-20 carbon atoms.

6. A method for preparing the water-soluble conjugated polymer for photothermal therapy according to claim 5, characterized in that, this method comprises the following steps: under the protection of inert gas, using an organic solvent to completely dissolve the polymeric monomer M according to claim 1 and the monomer containing the Ar structural unit, heating to 60° C. to 100° C. to incur the Suzuki polymerization reaction under the actions of a catalyst and tetraethylammonium hydroxide, with the reaction time being 12 to 36 hours; adding phenyl boronic acid, and continuing the reaction for 6 to 12 hours at a constant temperature; then adding bromobenzene, and continuing the reaction for 6 to 12 hours at a constant temperature; after the reaction stops, purifying the obtained reaction solution to obtain the target product.

7. The method for preparing the water-soluble conjugated polymer for photothermal therapy according to claim 6, characterized in that:
the organic solvent is at least one of dimethyl sulfoxide, tetrahydrofuran and N,N-dimethylformamide;
the catalyst is a system of palladium acetate and tricyclohexylphosphine or a system of tetrakis(triphenylphosphine) palladium;
the purification is operated specifically as follows: the obtained reaction solution is cooled to room temperature and then poured into acetone for precipitation, and then the obtained precipitate is filtered and dried to obtain a crude product; the crude product is extracted successively with methanol, acetone and n-hexane, then dissolved by using deionized water, settled out in an acetone solution for several times, filtered, and dried.

8. The method for preparing the water-soluble conjugated polymer for photothermal therapy according to claim 6, characterized in that:
the molar amounts of the polymeric monomer M and the monomer containing the Ar structural unit are equal; the amount of the catalyst is 5% to 5% by mol of the total amount of the reaction monomer; the amount of phenyl boronic acid is 10% to 20% by mol of the total amount of the reaction monomer; and the amount of bromobenzene is 2 to 5 times the molar amount of phenyl boronic acid.

9. Application of the water-soluble conjugated polymer for photothermal therapy according to claim 5 in the field of photothermal therapy.

10. Application of the water-soluble conjugated polymer for photothermal therapy according to claim 5 in preparation of a photothermal agent for photothermal therapy.
